(19) United States
(12) Patent Application Publication
(10) Pub. No.: US 2014/0275882 A1
(43) Pub. Date: Sep. 18, 2014

(54) Methods and Systems for Determining a Probe-Off Condition in a Medical Device

(71) Applicant: COVIDIEN LP, Mansfield, MA (US)

(72) Inventors: Paul Stanley Addison, Edinburgh (GB); James Nicholas Watson, Dunfermline (GB); Paul Mannheimer, Danville, CA (US)

(73) Assignee: Covidien LP, Mansfield, MA (US)

(21) Appl. No.: 13/797,832

(22) Filed: Mar. 12, 2013

(51) Int. Cl. A61B 5/1495; A61B 5/0205; A61B 5/1455

(52) U.S. Cl. CPC A61B 5/1495; A61B 5/14551; A61B 5/0205
USPC 600/324; 600/476

(57) ABSTRACT

A physiological monitoring system may use one or more characteristics of an ambient signal to determine a probe-off condition. A physiological sensor may be used to emit one or more wavelengths of light. A light signal may be received that includes an ambient light component and one or more components corresponding to the emitted light. One or more characteristics (e.g., baseline characteristics) of the ambient light component may be determined and compared to one or more thresholds. The system may determine whether the physiological sensor is properly positioned based on the comparison.

The present disclosure relates to determining a sensor condition, and more particularly relates to determining a probe-off condition in a pulse oximeter or other medical device.

### SUMMARY

Methods and systems are provided for determining whether a physiological sensor is properly positioned on a subject.

In some embodiments, a physiological monitoring system may emit, a photonic signal including one or more wavelengths of light. The system may receive the photonic signal after interaction with a subject. The system may process the received signal to determine one or more signals, including, for example, an ambient light signal. The ambient light signal may be processed to determine one or more characteristics. For example, the system may determine an ambient baseline characteristic, and may compare the ambient baseline characteristic to one or more thresholds. The system may determine a probe-off condition based on the comparison of one or more characteristics and one or more thresholds.

In some embodiments, the system, may use an ambient baseline level for determining whether a physiological sensor is properly positioned on a subject. For example, when an optical sensor begins to separate from a subject or become improperly positioned, the sensor may receive an increased amount of ambient light. The system may detect this increase in received ambient light and may determine that it is indicative of a probe-off or other improper condition.

In some embodiments, the system may use additional information for determining whether a physiological sensor is properly positioned on a subject. For example, changes in the received ambient light levels may be attributable to changes in the surrounding ambient light levels or changes in the sensor placement. By using additional information (e.g., a characteristic of received light corresponding to an emitted wavelength of light), the system can distinguish between improperly positioned sensors and changes in the surrounding ambient light level.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram, of an illustrative physiological monitoring system in accordance with some embodiments of the present disclosure;

FIG. 2A shows an illustrative plot of a light drive signal including a red light drive pulse and an IR light drive pulse in accordance with some embodiments of the present disclosure;

FIG. 2B shows an illustrative plot of a detector signal that may be generated by a sensor in accordance with some embodiments of the present disclosure;

FIG. 3 is a perspective view of an embodiment of a physiological monitoring system in accordance with some embodiments of the present disclosure;

FIG. 4 snows an illustrative signal processing system in accordance with, an embodiment, that may implement the signal processing techniques described herein;

FIG. 5 is a flow diagram showing illustrative steps for determining information about a physiological sensor in accordance with some embodiments of the present disclosure;

FIG. 6 is a panel showing two plots of illustrative system signals in accordance with some embodiments of the present disclosure; and

FIG. 7 is a panel showing two plots of illustrative system signals in accordance with some embodiments of the present disclosure.

### DETAILED DESCRIPTION OF THE FIGURES

The present, disclosure is directed towards determining a probe-off condition in a medical device. A physiological monitoring system may monitor one or more physiological parameters of a patient, typically using one or more physiological sensors. For example, the physiological monitoring system may include a pulse oximeter. The system may include, for example, a light source and a photosensitive detector. In some embodiments, a sensor may be attached to a target area of a patient. For example, the sensor may be attached using an adhesive, a strap, a band, elastic, any other suitable attachment, or any combination thereof. In some embodiments, the sensor may be located proximate to a desired structural element. For example, a sensor may be held near to the radial artery using a wrist strap. In another example, a sensor may be held near to the blood vessels of the forehead using an adhesive, tape, and/or a headband strap. In another example, a sensor may be held near the blood vessels on a fingertip using an adhesive, tape or clip.

In some embodiments, the system may determine a probe-off condition. As used herein, the probe-off condition may include any condition where the sensor is fully or partially detached or moved from the desired target area of the subject. A probe-off condition may include a condition where an adhesive coupling the sensor to the subject has fully or partially failed. A probe-off condition may include a condition where a sensor held with a strap or band has loosened, shifted, slid, moved, detached, repositioned in any other unsuitable arrangement, or any combination thereof. For example, a sensor held by an adhesive to the forehead of a subject may fully or partially separate due to an adhesive failure, resulting in a probe-off condition. In another example, a sensor held proximal to the radial artery at the wrist of a subject by a strap or band may shift out of position, resulting in a probe-off position. It will be understood that the probe-off conditions described here are merely exemplary and that any suitable undesirable positioning of the sensor may result in a probe-off condition. It will also be understood that the particular arrangement of a probe-off condition may dependent upon the configuration and type of probe.

The probe-off condition may be determined by the system. In some embodiments, the system may use an ambient light signal to determine a probe-off condition. As will be described in detail below, an ambient light signal may include the amount of light a detector receives when one or more associated light sources are in an “off” state. In some embodiments where a detector receives light from one or more light sources coupled to the system and from light sources not coupled to the system, the ambient light signal may include light from light sources not coupled to the system. Ambient light sources may include sunlight, incandescent room lights, fluorescent room lights, fireplaces, candles, naked flames, LED room lights, instrument panel lighting, heat sources, any other suitable light, sources not intended for determining a physiological parameter, or any combination thereof. It will be understood that heat sources may generate non-visible IR light that may be detected by the system. It will be understood that any visible or non-visible source of electromagnetic radiation may be included in the ambient light signal including, for example, radio waves, microwave, IR, visible, UV, X-ray, gamma ray. In some embodiments, the ambient light signal may include decaying LED light, from the system light sources. For example, it may take a particular amount of time for the light output from a light source to decrease to zero following the light drive signal being switched off. A portion of this emitted light may be included in the ambient signal. In some embodiments, the ambient light signal may not contain physiological information.

In some embodiments, a sensor may be designed to limit the amount, of ambient light received, by a detector. For example, a detector may be arranged close to and facing the skin. A detector may include a light blocking material between the detector and any ambient light sources, to prevent or significantly reduce ambient light from, reaching the detector. In a further example, a system may include other suitable shields, optics, filters, arrangements, or any combination thereof, to reduce ambient light, signals received by the receiver. In some embodiments, the particular arrangement of light blocking structures or material may depend on the type of probe. For example, a forehead probe may include flat light blocking structure, while a fingertip probe may include a light blocking structure that encircles the finger. However, some ambient light may still pass through the sensor and reach the detector. In addition, ambient light that is incident on the subject away from the sensor may pass through the subject's tissue and reach the detector.

It will be understood that many clinical settings include relatively bright light sources and the ambient light signals received by the detector may not be zero when the sensor is positioned as desired. Similarly, shielding ambient light may be more difficult for a forehead sensor than, for example, a fingertip sensor.

In some embodiments, for example, with a fingertip sensor where light, may be generated by the system, on one side of a finger and detected on the opposite side of a finger, removing the finger from the sensor (i.e., a probe-off condition) may result in all, or substantially all, of the generated light being received by the sensor, rather than a portion of the light, being attenuated by interacting with the tissue of the subject. This very high signal level may be determined by the system to be a probe-off condition.

In some embodiments, for example, with a forehead sensor, a probe-off condition may not result in a relatively nigh detected signal level. A forehead sensor may include a light source placed relatively close to a detector on the forehead of a patient using tape, an adhesive, a band encircling the skull, any other suitable arrangement, or any combination thereof. The light source and detector may be arranged such that a portion of the light emitted from the light source interacts with, and is partially attenuated by, the tissue of the subject and is detected by the detector. The light source may be pulsed, such that an ambient light signal is detected by the detector between the pulses, and a total signal detected during the pulses includes both the ambient and the desired light. In determining a physiological parameter, the ambient light signal may be, for example, subtracted from the total signal. In some embodiments, the ambient signal may exhibit, characteristic behavior of a probe-off condition. In some embodiments, the ambient light signal may remain relatively constant with respect to certain system changes. For example, the ambient light signal may be relatively insensitive to changes in physiological conditions.

In some embodiments, based, in part on the arrangement of the detector, a particular level of ambient signal may be considered by the system to be normal operation, whereas a higher level of ambient signal may be considered indicative of a probe-off condition. For example, where the detector is mostly shielded from ambient light, a low level ambient signal may be considered normal, and an ambient signal above a threshold may be considered indicative of a probe-off condition. For example, the high ambient light signal may be associated with the detector separating from the patient and receiving more light from a room lighting source. The detector may fall off or otherwise detach, from, a patient and be disposed facing an ambient light source. In some embodiments, the detector may be positioned such that it is receiving more light generated by the light source of the system that is not attenuated, by the tissue of the subject. In some embodiments, a threshold may be useful in identifying a probe-off condition in a situation where the probe is slowly detaching from a surface. For example, the threshold may be useful in slowly evolving, dynamic transitions from a properly positioned probe to a probe-off condition.

In some embodiments, the ambient signal may be compared to a threshold level to determine a probe-off condition. In some embodiments, a trend, slope, or other derivative of the ambient signal level may be compared to a target or desired parameter to determine a probe-off condition. In some embodiments, the level or trend of a light pulse signal level may be compared to an ambient signal to determine a probe-off condition. In some embodiments, the threshold may be set by a user, may be predetermined, may be set based on historical data, may be set based on any other suitable information, or any combination thereof. In some embodiments, the threshold may be determined during a reset period.

In some embodiments, the system, may compare a first signal level to a second signal level. For example, a level or trend of the ambient signal may be compared, to a level or trend, of a drive pulse signal. In some embodiments, the drive pulse signal may include light from a red light emitting diode, an infrared light emitting diode, any other suitable light emitter, or any combination thereof. In some embodiments, the system may combine signals before or after comparing signals.

It will be understood that any of the aforementioned signal levels, thresholds, and targets may be used in any suitable combination. For example, a change in the level of the ambient light (e.g., an examination light is switched on) may be distinguished from a probe-off condition by comparing multiple signal levels, by a trend, by a rate of change, by user input, by any other suitable technique, or any combination thereof.

An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot, hand, other suitable body part, or any combination thereof. The oximeter may use a light source to pass light, through blood, perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid, artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount, of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is selected to be of one or more wavelengths that are attenuated by the blood in an amount representative of the blood constituent concentration. Red and infrared (IR) wavelengths may be used because it has been observed that highly oxygenated blood will absorb relatively less red light and more IR light than blood with, a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine blood oxygen saturation using two wavelengths of light and a ratio-of-ratios calculation. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to determine ambient signal levels, reduce overlap of the light drive pulses, allow time for light sources to stabilize, allow time for the detector to stabilize, allow time for the detected light signal to stabilize, reduce heating effects, reduce power consumption, for any other suitable reason, or any combination thereof.

It will be understood that the probe-off techniques described herein are not limited to pulse oximeters and may be applied to any suitable medical and non-medical devices. For example, the system may include probes for regional saturation (rSO2), respiration rate, respiration effort, continuous non-invasive blood pressure, saturation pattern detection, fluid responsiveness, cardiac output, any other suitable clinical parameter, or any combination thereof. Probes may be used with a pulse oximeter, a general purpose medical monitor, any other suitable medical device, or any combination thereof. In some embodiments, the probe-off identification techniques described herein may be applied to analysis of light levels where an ambient or dark signal is used.

The following description and accompanying FIGS. 1-7 provide additional details and features of some embodiments of determining a sensor condition in a medical device.

FIG. 1 is a block diagram of an illustrative physiological monitoring system 100 in accordance with some embodiments of the present disclosure. Physiological monitoring system 100 may include a sensor 102 and a monitor 104 for generating and processing physiological signals of a subject. In some embodiments, sensor 102 and monitor 104 may be part of an oximeter.

Sensor 102 of physiological monitoring system 100 may include light source 130 and detector 140. Light source 130 may be configured to emit photonic signals having one or more wavelengths of light (e.g. Red and IR) into a subject's tissue. For example, light, source 130 may include a Red light emitting light source and an IR light emitting light source, e.g. Red and IR light emitting diodes (LEDs), for emitting light into the tissue of a subject, to generate physiological signals. In one embodiment, the Red wavelength may be between about 600 nm and about 700 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a Red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the Red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, in a transmission arrangement, where the detector 140 is on the other side of a body part from light source 130, when more light at a certain wavelength is absorbed, less light of that wavelength is typically received from the tissue by detector 140. In a reflection arrangement, where both light source 130 and detector 140 are on the same side of a body part, the increased reflection of light may result in a higher detected light level. After converting the received light to an electrical signal, detector 140 may send the detection signal to monitor 104, where the detection signal may be processed and physiological parameters may be determined (e.g., based on the absorption of the Red and IR wavelengths in the subject's tissue). In some embodiments, the detection signal may be preprocessed by sensor 102 before being transmitted to monitor 104.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals of control circuitry 110 to operate synchronously with light drive circuitry 120. For example, front end processing circuitry 150 may synchronize the operation of an analog-to-digital converter and a demultiplexer with, the light drive signal based on the timing control signals. In addition, the back end processing circuitry 170 may use the timing control signals of control circuitry 110 to coordinate operation with front end processing circuitry 150.

Light drive circuitry 120, as discussed above, may be configured to generate a light drive signal that is provided to light source 130 of sensor 102. The light drive signal may, for example, control the intensity of light source 130 and the timing of switching light source 130 on and off. When light source 130 is configured to emit, two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

FIG. 2A shows an illustrative plot of a light drive signal including red drive pulse 202 and IR drive pulse 204 in accordance with some embodiments of the present disclosure. Red drive pulse 202 and IR drive pulse 204 may be generated by light drive circuitry 120 under the control of control circuitry 110. As used herein, drive pulses may refer to switching power or other components on and off, high and low output states, high and low values within a continuous modulation, other suitable relatively distinct states, or any combination thereof. The light drive signal may be provided to light source 130, including red drive pulse 202 and IR drive pulse 204 to drive red and IR light emitters, respectively, within light source 130. Red drive pulse 202 may have a higher amplitude than IR drive pulse 204 since red LEDs may be less efficient than IR LEDs at converting electrical energy into light energy. In some embodiments, the output levels may be the equal, may be adjusted for nonlinearity of emitters, may be modulated in any other suitable technique, or any combination thereof. Additionally, red light may be absorbed and scattered more than IR light when passing through perfused tissue. When the red and IR light sources are driven in this manner they emit pulses of light at their respective wavelengths into the tissue of a subject in order generate physiological signals that physiological monitoring system 100 may process to calculate physiological parameters. It will be understood that the light drive amplitudes of FIG. 2A are merely exemplary any that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the Red and IR light drive pulse. “Off” periods 220 are periods during which no drive current may be applied to light source 130. “Off” periods 220 may be provided, for example, to prevent overlap of the emitted light, since the light drive signal provided to light source 130 may require time to turn completely on and completely off. The period from time point 216 to time point 218 may be referred to as a drive cycle, which includes four segments in FIG. 2A: a red drive pulse 202, followed by an “off” period 220, followed by an IR drive pulse 204, and followed by an “off” period 220. After time point 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red drive pulse 202 and each IR drive pulse 204 may be understood to be surrounded by two “off” periods 220 in FIG. 2A. “Off” periods may also be referred to as dark periods, in that the emitters are dark during that period.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current waveform 214 may be generated in response to a light source being driven by the light drive signal of FIG. 2A. The valleys of detector current waveform 214 may be synchronous with periods of time during which no light is being emitted by the light source. While no light is being emitted by a light source during the valleys, detector current waveform 214 may not decrease to zero. Rather, ambient signal 222 may be present in the detector waveform, as well as other background amplitude contributions. In some embodiments, detector current waveform 214 may be distorted from an ideal square wave due to pulse shaping effects of switching in the light drive signal, switching of the light emitters, switching in the detector, parasitic capacitance, any other suitable effects, or any combination thereof. For example, the signal from detector 140 may require time to decay completely to its final state after light source 130 is switched off. In some embodiments, ambient signal 222 may be used to determine a probe-off condition. In some embodiments, ambient signal 222 may be removed from a processed signal to facilitate determination of physiological parameters.

Referring back to FIG. 1, front end processing circuitry 150, which may receive a detection signal, such as detector current waveform 214, may include analog conditioning 152, demultiplexer 154, digital conditioning 156, analog-to-digital converter 158, decimator/interpolator 160, and ambient subtractor 162.

In some embodiments, front end processing circuitry 150 may include another analog-to-digital converter (not shown) configured to sample the unprocessed detector signal. This signal may be used to detect changes in the ambient light level without applying the signal condition and other steps that may improve the quality of determined physiological parameters but may reduce the amount of information regarding a probe-off condition.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

The conditioned analog signal may be processed by analog-to-digital converter 158, which may convert the conditioned analog signal into a digital signal. Analog-to-digital converter 158 may operate under the control of control circuitry 110. Analog-to-digital converter 158 may use timing control signals from control circuitry 110 to determine when to sample the analog signal. Analog-to-digital converter 158 may be any suitable type of analog-to-digital converter of sufficient resolution to enable a physiological monitor to accurately determine physiological parameters.

Demultiplexer 154 may operate on the analog or digital form of the detector signal to separate out different components of the signal. For example, detector current, waveform 214 of FIG. 2B includes a Red component, an IR component, and at least one ambient component. Demultiplexer 154 may operate on detector current waveform 214 of FIG. 2B to generate a Red signal, an IR signal, a first ambient signal (e.g., corresponding to the ambient component that occurs immediately after the Red component), and a second ambient signal (e.g., corresponding to the ambient component that occurs immediately after the IR component). Demultiplexer 154 may operate under the control of control circuitry 110. For example, demultiplexer 154 may use timing control signals from control circuitry 110 to identify and separate out the different components of the detector signal.

Digital conditioning 156 may perform any suitable digital conditioning of the detector signal. The digital conditioning may include any type of digital filtering of the signal (e.g., low pass, high pass, band pass, notch, or any other suitable filtering), amplifying, performing an operation on the signal, performing any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove ambient values from the Red and IR components. In some embodiments, the system may subtract the ambient values from the Red and IR components to generate adjusted Red and IR signals. For example, ambient subtractor 162 may determine a subtraction amount from the ambient signal portion of the detection signal and subtract it from the peak portion of the detection signal in order to reduce the effect of the ambient signal on the peak. For example, in reference to FIG. 2A, a detection signal peak corresponding to red drive pulse 202 may be adjusted by determining the amount of ambient signal during the “off” period 220 preceding red drive pulse 202. The ambient signal amount determined in this manner may be subtracted from the detector peak corresponding to red drive pulse 202. Alternatively, the “off” period 220 after red drive pulse 202 may be used to correct red drive pulse 202 rather than the “off” period 220 preceding it. Additionally, an average of the “off” periods 220 before and after the “on” period of red drive pulse 202 may be used. In some embodiments, ambient subtractor 162 may output an ambient signal for further processing. Ambient subtractor 162 may average the ambient signal from multiple “off” periods 220, may apply filters to the ambient signal such as averaging filters, integration filters, delay filters, buffers, counters, any other suitable filters or processing, or any combination thereof.

It will be understood that in some embodiments, ambient subtractor 162 may be omitted. It will also be understood that in some embodiments, the system may not subtract the ambient contribution of the signal. It will also be understood that the functions of demultiplexer 154 and ambient subtractor 162 may be complementary, overlapping, combined into a signal function, combined or separated in any suitable arrangement, or any combination thereof. For example, the received light signal may include an ambient signal, an IR light signal, and a red light signal. The system may use any suitable arrangement of demultiplexer 154 and ambient subtractor 162 to determine or generate any combination of: a red signal, an IR signal, an red ambient signal, an IR ambient signal, an average ambient signal, a red+ambient signal, an IR+ambient signal, any other suitable signal, or any combination thereof.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 158. This may be achieved by applying gain to the detection signal by analog conditioning 152 to map the expected range of the detection signal to the full or close to full output range of analog-to-digital converter 158. The output value of analog-to-digital converter 158, as a function of the total analog gain applied to the detection signal, may be given as:

ADC Value=Total Analog Gain×[Ambient Light+LED Light].

Ideally, when ambient light is zero and when the light source is off, the analog-to-digital converter 158 will read just above the minimum input value. When the light source is on, the total analog gain may be set such, that the output of analog-to-digital converter 158 may read close to the full scale of analog-to-digital converter 158 without saturating. This may allow the full dynamic range of analog-to-digital converter 158 to be used, for representing the detection signal, thereby increasing the resolution of the converted signal. In some embodiments, the total analog gain may be reduced by a small amount so that small changes in the light level incident on the detector do not cause saturation of analog-to-digital converter 158.

Back end processing circuitry 170 may include processor 172 and memory 174. Processor 172 may be adapted to execute software, which may include an operating system, and one or more applications, as part of performing the functions described herein. Processor 172 may receive and further process physiological signals received from front end processing circuitry 150. For example, processor 172 may determine one or more physiological parameters based on the received physiological signals. Memory 174 may include any suitable computer-readable media capable of storing information that can be interpreted by processor 172. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled, with user interface 180 and communication interface 190.

User interface 180 may include user input 182, display 184, and speaker 186. User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth. In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (referred to as an “SpO₂” measurement), pulse rate information, respiration rate information, blood pressure, sensor condition, any other parameters, and any combination thereof. Display 184 may include any type of display such as a cathode ray tube display, a flat panel display such a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communication interface 190 may include any suitable hardware, software, or both, which may allow monitor 104 to communicate with electronic circuitry, a device, a network, a server or other workstations, a display, or any combination thereof. Communication interface 190 may include one or more receivers, transmitters, transceivers, antennas, plug-in connectors, ports, communications buses, communications protocols, device identification protocols, any other suitable hardware or software, or any combination thereof. Communication interface 190 may be configured to allow wired communication (e.g., using USB, RS-232, Ethernet or other standards), wireless communication (e.g., using WiFi, IR, WiMax, BLUETOOTH, UWB, or other standards), or both. For example, communication interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communication interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In some embodiments, all of the components of physiological monitoring system 100 can be realized in processor circuitry.

FIG. 3 is a perspective view of an embodiment of a physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. For example, in a transmission arrangement such, as a fingertip clip sensor, light, source 316 and detector 318 may be on opposing sides of a finger, and light will be transmitted, through the finger, partially attenuated by tissue and pulsatile blood flow. In another example, in a reflection arrangement such as a forehead sensor, light travels through the subject's tissue from light source 316 to detector 318. Any suitable configuration of light source 316 and detector 318 may be used. In an embodiment, sensor unit 312 may include multiple light sources and detectors, which may be spaced, apart. Physiological monitoring system 310 may also include one or more additional sensor units (not shown) that may, for example, take the form of any of the embodiments described here in with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two or more different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected to monitor 314 (not shown). Monitor 314 may be configured to calculate physiological parameters based, at least in part on data relating to light emission and detection received from one or more sensor units such as sensor unit 312. For example, monitor 314 may be configured to determine pulse rate, blood, pressure, blood, oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range or when a sensor is not properly positioned. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324 through port 336. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The multi-parameter physiological monitor 326 may include a display 328 including a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide for information from monitor 314 and from other medical monitoring devices or systems (not shown) using, for example, display 328. For example, multi-parameter physiological monitor 326 may be configured to display an estimate of a subject's blood oxygen saturation and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input, port, or a digital communications port, respectively and/or may communicate wirelessly (not shown). In addition, monitor 314 and/or multi-parameter physiological monitor 326 may be coupled to a network to enable the sharing of information with servers or other workstations (not shown), Monitor 314 may be powered by a battery (not shown) or by a conventional power source such as a wall outlet.

In some embodiments, all or some of monitor 314 and multi-parameter physiological monitor 326 may be referred to collectively as processing equipment. In some embodiments, any of the processing components and/or circuits, or portions thereof, of FIGS. 1, 3 and 4 (below) may be referred to collectively as processing equipment. For example, processing equipment may be configured to generate light drive signals, amplify, filter, sample and digitize detector signals, and calculate physiological information from the digitized signal. In some embodiments, all or some of the components of the processing equipment may be referred to as a processing module.

FIG. 4 snows illustrative signal processing system 400 in accordance with an embodiment that may implement the signal processing techniques described herein. Signal processing system 400 includes input signal generator 410, processor 412 and output 414. In the illustrated embodiment, input signal generator 410 may include pre-processor 420 coupled to sensor 418. As illustrated, input signal generator 410 generates an input signal 416. In some embodiments, input signal 416 may include one or more intensity signals based on a detector output. In some embodiments, pre-processor 420 may be an oximeter and input signal 416 may be a PPG signal. In an embodiment, pre-processor 420 may be any suitable signal processing device and input signal 416 may include PPG signals and one or more other physiological signals, such as an electrocardiogram (EGG) signal. It will be understood that input signal generator 410 may include any suitable signal source, signal generating data, signal generating equipment, or any combination thereof to produce input signal 416. Input signal 416 may be a single signal, or may be multiple signals transmitted over a single pathway or multiple pathways.

Pre-processor 420 may apply one or more signal processing operations to the signal generated by sensor 418. For example, pre-processor 420 may apply a pre-determined set of processing operations to the signal provided by sensor 418 to produce input signal 416 that can be appropriately interpreted by processor 412, such as performing A/D conversion. In some embodiments, A/D conversion may be performed by processor 412. Pre-processor 420 may also perform any of the following operations on the signal provided by sensor 418: reshaping the signal for transmission, multiplexing the signal, modulating the signal onto carrier signals, compressing the signal, encoding the signal, and filtering the signal. In some embodiments, pre-processor 420 may include a current-to-voltage converter (e.g., to convert a photocurrent into a voltage), an amplifier, a filter, and A/D converter, a de-multiplexer, any other suitable pre-processing components, or any combination thereof. In some embodiments, pre-processor 420 may include one or more components from front end processing circuitry 150 of FIG. 1.

In some embodiments, input signal 416 may include PPG signals corresponding to one or more light frequencies, such as an IR PPG signal and a Red PPG signal, and ambient light. In some embodiments, input signal 416 may include signals measured at one or more sites on a subject's body, for example, a subject's finger, toe, ear, arm, or any other body site. In some embodiments, input signal 416 may include multiple types of signals (e.g., one or more of an ECG signal, an EEG signal, an acoustic signal, an optical signal, a signal representing a blood pressure, and a signal representing a heart rate). Input signal 416 may be any suitable biosignal or any other suitable signal.

In some embodiments, input signal 416 may be coupled to processor 412. Processor 412 may be any suitable software, firmware, hardware, or combination thereof for processing input signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may, for example, include an assembly of analog electronic components. Processor 412 may calculate physiological information. For example, processor 412 may compute one or more of a pulse rate, respiration rate, blood pressure, or any other suitable physiological parameter. Processor 412 may perform, any suitable signal processing of input signal 416 to filter input signal 416, such as any suitable band-pass filtering, adaptive filtering, closed-loop filtering, any other suitable filtering, and/or any combination thereof. Processor 412 may also receive input signals from, additional sources (not shown). For example, processor 412 may receive an input signal, containing information about treatments provided to the subject. Additional input signals may be used, by processor 412 in any of the calculations or operations it performs in accordance with signal processing system 400.

In some embodiments, all or some of pre-processor 420, processor 412, or both, may be referred to collectively as processing equipment. In some embodiments, any of the processing components and/or circuits, or portions thereof, of FIGS. 1, 3, and 4 may be referred to collectively as processing equipment. For example, processing equipment may be configured to amplify, filter, sample and digitize input signal 416 (e.g., using an analog-to-digital converter), and calculate physiological information from the digitized signal. In some embodiments, all or some of the components of the processing equipment may be referred to as a processing module.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store fiducial information or initialization information corresponding to physiological monitoring. In some embodiments, processor 412 may store physiological measurements or previously received data from input signal 416 in a memory device for later retrieval. In some embodiments, processor 412 may store calculated values, such as a pulse rate, a blood pressure, a blood oxygen saturation, a fiducial point location or characteristic, an initialization parameter, or any other calculated values, in a memory device for later retrieval.

Processor 412 may be coupled to output 414. Output 414 may be any suitable output device such as one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that signal processing system 400 may be incorporated into physiological monitoring system 100 of FIG. 1 in which, for example, input, signal generator 410 may be implemented as part of sensor 102, or into physiological monitoring system 310 of FIG. 3 in which, for example, input signal generator 410 may be implemented as part of sensor unit 312 of FIG. 3, and processor 412 may be implemented as part of monitor 104 of FIG. 1 or as part of monitor 314 of FIG. 3. Furthermore, all or part of signal processing system 400 may be embedded in a small, compact, object carried with or attached to the subject, (e.g., a watch, other piece of jewelry, or a smart, phone). In some embodiments, a wireless transceiver (not shown) may also be included in signal processing system 400 to enable wireless communication with other components of physiological monitoring systems 100 of FIGS. 1 and 310 of FIG. 3. As such, physiological monitoring systems 100 of FIGS. 1 and 310 of FIG. 3 may be part of a fully portable and continuous subject monitoring solution. In some embodiments, a wireless transceiver (not shown) may also be included in signal processing system 400 to enable wireless communication with other components of physiological monitoring systems 100 of FIGS. 1 and 310 of FIG. 3. For example, pre-processor 420 may transmit input signal 416 over BLUETOOTH, 802.11, WiFi, WiMax, cable, satellite, Infrared, or any other suitable transmission scheme. In some embodiments, a wireless transmission scheme may be used, between any communicating components of signal processing system 400. In some embodiments, signal processing system 400 may include one or more communicatively coupled, modules configured to perform particular tasks. In some embodiments, signal processing system 400 may be included as a module communicatively coupled, to one or more other modules.

It will be understood that the components of signal processing system 400 that are shown and described as separate components are shown and described as such for illustrative purposes only. In other embodiments the functionality of some of the components may be combined in a single component. For example, the functionality of processor 412 and pre-processor 420 may combined in a single processor system. Additionally, the functionality of some of the components shown and described herein may be divided over multiple components. Additionally, signal processing system 400 may perform the functionality of other components not show in FIG. 4. For example, some or all of the functionality of control circuitry 110 of FIG. 1 may be performed in signal processing system 400. In other embodiments, the functionality of one or more of the components may not be required. In an embodiment, all of the components can be realized in processor circuitry.

FIG. 5 is flow diagram 500 showing illustrative steps for determining information about a physiological sensor in accordance with some embodiments of the present disclosure.

In step 502, the system may use the physiological sensor to emit a photonic signal. The system may emit a photonic signal including one wavelength of light, multiple wavelengths of light, a broad spectrum light, (e.g., white light), or any combination thereof. For example, the photonic signal may include light from a red LED and light from an IR LED. The emitted photonic signal may be emitted, for example, by light source 130 of FIG. 1. In some embodiments, the emitted photonic signal may include a light drive modulation. For example, when the photonic signal includes a red light source and an IR light source, the light drive modulation may include a red drive pulse followed by an “off” period followed by an IR drive pulse followed by an off period. It will be understood that this drive cycle modulation is merely exemplary and that any suitable drive cycle modulation or combination of modulations may be used. In some embodiments, the photonic signal may include a cardiac cycle modulation, where the brightness, duty cycle, or other parameters of one or more emitters are varied at a rate substantially related to the cardiac cycle.

In step 504, the system, may receive a light signal. The received light signal may include light from drive pulses or other emitted light in the emitted, photonic signal that has interacted with the subject. The received light signal may be detected by, for example, detector 140 of FIG. 1. In some embodiments, a portion of the emitted light may be partially attenuated by the tissue of the subject before being received, as a received, light, signal. In some embodiments, the received light may have been primarily reflected by the subject. For example, reflected light may be detected by a forehead-attached system where the emitter and detector are on the same side of the subject. In some embodiments, the received light may have been transmitted through the subject. For example, transmitted light may be detected in a fingertip-attached or earlobe-attached sensor.

In some embodiments, the received, light, signal may include an ambient light signal component and a component related to the emitted photonic signal. In some embodiments, the received light signal, may include a first wavelength light component, a second wavelength light component, and an ambient signal light component. The components may be combined and multiplexed in any suitable arrangement. The first and second wavelength light components may be the component of the received signal related, to the emitted photonic signal, where the emitted photonic signal includes a first and second wavelength. The ambient light component may be demultiplexed, for example, from the received light signal during the period of a light drive cycle when the emitters are not emitting light. For example, the ambient light component may relate to “off” periods 220 of FIG. 2 and the component related to the emitted photonic signal may relate to the signal received during a drive pulse, such, as red drive pulse 202 of FIG. 2.

In some embodiments, the ambient light component, may, for example, correspond to ambient signal 222 of FIG. 2. In some embodiments, the system may subtract ambient signal 222 or a signal derived from ambient signal 222 from the received signal to generate an adjusted signal. The adjusted signal may be used to determine physiological parameters. In some embodiments, the system may extract from a received light signal an ambient signal for probe-off analysis before generating the adjusted signal. Separation of the ambient signal from the received signal may include, for example, using demultiplexer 154 of FIG. 1. In some embodiments, the system may apply ambient subtractor 162 of FIG. 1 to a demultiplexed signal that will be used to determine physiological parameters, but will not apply ambient subtractor 162 of FIG. 1 to the ambient signal. Signal processing of the ambient component and emitted light component may include any suitable components of physiological monitoring system 100 of FIG. 1, physiological monitoring system 310 of FIG. 3, any other suitable components, or any combination thereof.

In some embodiments, the system may adjust or compensate a signal depending in part on the LED drive signal, the detector gain, other suitable system parameters, or any combination thereof. For example, increasing the gain on a detected signal may result in an increased ambient signal. The system may compensate for this increase that is not correlated with, a change in the sensor position. In a further example, the system may change the LED emitter brightness, resulting in a change in the detected signals. The system may compensate for these changes in the detected signal amplitude to distinguish them from a change in the sensor positron. It will be understood that, the system may make any adjustments in gain, amplification, frequency, wavelength, amplitude, any other suitable adjustments, or any combination thereof. It will be understood that the adjustments may be made to the emitted photonic signal, the received signal, a signal following a number of processing steps, any other suitable signals, or any combination thereof.

In step 506, the system may determine a characteristic of the light signal. The characteristic may be a baseline characteristic of the ambient light component of the received light signal. The baseline characteristic may include the signal level, amplitude, rate of change, slope, moving average, other trend, any other suitable characteristic, or any combination thereof. A trend may include, for example, a first derivative of the amplitude signal. A baseline characteristic may include a combination of parameters. For example, a trend may include the magnitude and polarity of the first derivative. In another example, the baseline characteristic may include the signal amplitude and the polarity of the first derivative. Baseline characteristics may be relative, absolute, or any combination thereof. For example, the signal level may be the absolute amplitude. In another example, the signal level may be relative to an ambient signal or to another signal. Determining the signal level may include any suitable processing equipment described above. The system may apply filtering, smoothing, averaging, any other suitable technique, or any combination thereof. For example, the ambient signal may be filtered to remove noise. In another example, the ambient signal, and any other signal, may be smoothed or averaged to remove transient signals.

In step 508, the system may compare the characteristic (e.g., a baseline characteristic) to a threshold. In some embodiments, the system may include one or more threshold levels related to the signal characteristic. Reaching or crossing a threshold may result in an alarm being triggered, a flag being set, an indication being generated, a signal being generated, any other suitable output, or any combination thereof. Thresholds may be predetermined, set by the user, determined based on historical information, determined based on characteristics related to the patient, determined based on characteristics of the sensor and system, determined based on any other suitable criteria, or any combination thereof. Thresholds may be constant or vary in time. The threshold may include multiple threshold values corresponding to multiple characteristics. In some embodiments, the threshold may be adjusted or compensated based on system gain changes (e.g., a detector gain change).

In some embodiments, the threshold may be set during a reset period. For example, the reset period may be triggered by a user to indicate a normal operating state of the system. The normal operating state may include proper positioning of the sensor. The reset mode may include setting a normal baseline characteristic, for the ambient signal (e.g., ambient level or trend) and determining a threshold based on the normal baseline characteristic. In some embodiments, a reset period may be triggered automatically based on time, sensor connections, signal conditions, a physiological condition or event, any other suitable triggers, or any combination thereof.

In some embodiments, where the baseline characteristic is an ambient signal level, the threshold may be an upper limit on ambient signal level before an output is triggered. For example, an upper limit may be set such that the system may detect when the detector is receiving a more than expected amount, of ambient light. In some embodiments, a low threshold may be indicative of a detector problem or other system parameters. The threshold may be a constant level, a moving average, a predetermined pattern, a pattern determined based on user input, a pattern based on historical information, any other suitable threshold, or any combination thereof.

In some embodiments, where the baseline characteristic is a trend of the ambient signal, the threshold may be an upper or lower limit, on the trend. The trend may include a first, second, or higher derivative of the ambient signal. The trend, may include a moving average, integrated value, any other suitable trend, or any combination thereof. For example, the trend may be the slope of the signal and the threshold may be an upper and/or lower slope value. In another example, the trend may be a moving average. The threshold may be a constant level, a moving average, a predetermined pattern, a pattern determined based, on user input, a pattern based on historical information, any other suitable threshold, or any combination thereof.

In some embodiments, the characteristic may include a comparison between multiple signals. For example, the system may identify a condition where the ambient signal, increases while another signal component remains relatively constant. In another example, the system may identify a condition where the ambient signal increases and another signal component decreases by a similar amount. For example, a large increase in ambient light caused by switching on an examination room light source may cause the ambient light signal to cross a threshold. Comparing the ambient signal to another signal may help to classify an ambient signal change. In another example, an external detector, for example, on the monitor, may be used to determine an ambient light level that could be used to normalize changes in the detected ambient light signal of the sensor. In some embodiments, the ambient signal level may be compared to the signal component related to the emitted light, a processed light signal, any other suitable signal, or any combination thereof. The comparison may include a subtraction, division, multiplication, integration, any other suitable function, or any combination thereof. Comparisons may also include time-domain comparisons. For example, an ambient signal level may be compared to the moving average of the emitted light related component. In some embodiments, comparing multiple signals may help identify a probe-off or other undesirable system condition from an external, unrelated change.

In step 510, the system may determine whether the sensor is properly positioned. The system may determine whether the sensor is properly positioned based the comparison of the signal characteristic to the threshold. For example, if the signal characteristic is an ambient signal level and the level is above a threshold, the system may determine that the sensor is in a probe-off condition. For example, the system may receive a relatively high level of ambient light when a detector is detaching or detached from a subject and is thus less shielded from ambient light. In some embodiments, for example when the light, is transmitted through the subject and received by a fingertip sensor, a probe-off condition may result in a nigh emitted light component level as compared to the ambient component level in the received signal. In some embodiments, for example where the ambient signal characteristic is a trend, the slope crossing a high or low threshold may be indicative of a probe-off condition because of the high rate of change. In some embodiments, thresholds or comparisons may be used to distinguish between a slowly detaching sensor and a rapidly detaching sensor.

In some embodiments, the system may use multiple criteria to determine a probe-off condition. The multiple criteria may be combined using any suitable logic method, algorithmic method, polling method, weighted method, any other suitable methods, or any combination thereof. In some embodiments, the system, may determine a confidence value related to the possibility of a probe-off condition based on the criteria.

It will be understood that the above described probe-off detection techniques are merely exemplary and that any suitable signal characteristics or combination of signal characteristics may be used with any suitable thresholds or combination of thresholds to determine a probe-off condition.

FIG. 6 is a panel showing illustrative system signal plots 600 and 620 in accordance with some embodiments of the present disclosure.

Plot 600 may include light component 602 and ambient signal 604. The abscissa axis of plot 600 may be in units of time, and the ordinate axis may be in units of amplitude. Light, component 602 and ambient signal 604 may be on the same or difference amplitude scales. In some embodiments, the signals may be scaled, shifted, normalized, processed by any other suitable technique, or any combination thereof. The amplitude of the signals in plot 600 may relate to the intensity of the related received light signal.

Light component 602 may include, for example, information from one wavelength of light, from a drive cycle modulation and ambient light. For example, light component 602 may include information related to an IR light drive signal and the ambient light. In another example, light component 602 may include information related to a red light drive signal and the ambient light. In another example, light component 602 may contain information from multiple wavelengths of light. In some embodiments, light component 602 may include the component related to the emitted photonic signal received in step 504 of FIG. 5.

Ambient signal 604 may include, for example, information from the “off” periods of a drive pulse modulation. The ambient signal level may correspond to, for example, ambient signal 222 of FIG. 2A. In some embodiments, ambient signal 604 may correspond to the ambient component received in step 504 of FIG. 5. The level of the ambient signal may relate to the intensity of light received by the detector related to light, not emitted from the sensor, for example, light from room light, sunlight, instrument lights, any other suitable source, or any combination thereof.

Plot 600 may relate to received, signals during a slow detaching of a forehead sensor. For example, the period of time depicted in plot 600 may be approximately 1 minute. Region 606 of plot 600 may be a region corresponding to a normal sensor position. For example, a sensor in a proper position may detect a moderate light component signal level, as indicated by light component 602, and a low ambient signal level, as indicated by ambient signal 604. During the time indicated by region 608, a sensor may begin to slowly detach. In the illustrated example, the emitter may begin to detach from the subject before the detector begins to detach. Thus, the light component signal level may decrease while the ambient light signal remains constant. During the time indicated by region 610, both the emitter and the detector may be slowly detaching from the subject, exposing the detector to more ambient light. During region 610, the level of ambient, signal 604 may increase as well as light component 602. The increase in light, component 602 may relate to, for example, emitted light reaching the sensor without being attenuated by the subject's tissue. During region 612, the sensor may detach fully from the patient. The unstable and decreased amplitude in both signal levels may relate to the sensor being located on, for example, the subject's bed sheets or clothes. In region 614, the sensor may, for example, be laying sensor-side up on a table or floor. This may result in only ambient light, reaching the detector. Thus, the amount of light detected during light “on” periods may be similar or equal to the amount of light detected during “off” periods of a drive cycle modulation.

Plot 620 may include a logic flag set when a potential probe-off condition is detected. The abscissa axis of plot 620 may be in units of time, and the ordinate axis may be in binary logic values 0 and 1. In some embodiments, flag signal 622 may be indicative of a flag or logic value set when a potential probe-off condition is identified. For example, when an ambient signal characteristic is compared to a threshold in step 508 of FIG. 5, the result of the comparison may be used to set flag signal 622 to 0 or 1. In some embodiments, an ambient signal level above a threshold may result in flag signal being set to 1.

In the embodiment illustrated in FIG. 6, flag signal 622 may be set to 1 at time point 616. Time point 616 may be indicative of when ambient signal 604 crosses threshold 618. Region 626 may include the flag signal 622 at 0, indicative of the sensor being in a desirable position. Region 628, including flag signal 622 at 1, may be indicative of the sensor being in an undesirable position. In some embodiments, flag signal 622 may remain at 1 until reset to 0 by a user, reset by the ambient signal falling below threshold 618, reset following a duration of time, reset based on the amplitude of the ambient signal exceeding threshold 618, reset based on any other suitable input or signal, or any combination thereof.

It will be understood that the time intervals indicated by regions 606, 608, 610, 612, 614, 626, and 628 are both approximate and exemplary. Similarly, the particular location of time point 616 is both approximate and exemplary. For example, the boundary between region 606 and region 608 may be slightly earlier or later in time. In another example, the specific transition from region 610 to region 612 is approximated in plot 600. It will also be understood that the regions may overlap, be non-contiguous, be in any other suitable arrangement, or any combination thereof.

FIG. 7 is a panel showing illustrative system signal plots 700 and 720 in accordance with some embodiments of the present disclosure.

Plot 700 may include light component 702 and ambient signal 704. The abscissa axis of plot 700 may be in units of time, and the ordinate axis may be in units of amplitude. Light component 702 and ambient signal 704 may be on the same or difference amplitude scales. In some embodiments, the signals may be scaled, shifted, normalized, processed by any other suitable technique, or any combination thereof. The amplitude of the signals in plot 700 may correspond to the intensity of the related portions of the received light signal.

Light component 702 may include, for example, information from one wavelength of light from a drive cycle modulation and ambient light. For example, light component 702 may include information related to an IR light drive signal and the ambient light. In another example, light component 702 may include information related to a red light drive signal and the ambient light. In another example, light component 702 may contain information from multiple wavelengths of light. In some embodiments, light component 702 may include the component related to the emitted photonic signal received in step 504 of FIG. 5.

Ambient signal 704 may include, for example, information from the “off” periods of a drive pulse modulation. The ambient signal level may correspond to, for example, ambient signal 222 of FIG. 2A. In some embodiments, ambient signal 704 may include the ambient component received in step 504 of FIG. 5. The level of the ambient signal may relate to the intensity of light received by the detector related to light not emitted from the sensor, for example, light from room light, sunlight, instrument lights, any other suitable source, or any combination thereof.

Plot 700 may relate to received signals when the amount of ambient light increases. In some embodiments, the level of ambient light may increase with the sensor properly positioned. The system, may use the signals in plot 700 to determine that the increase in ambient light is not indicative of a probe-off condition. For example, the period of time depicted in plot 700 may be approximately 1 minute. In region 706, the ambient room lighting may be at a first, relatively lower level and in region 708, the ambient room lighting may be at a second, relatively higher level. For example, a bright light may be switched, on in the room at time point 710.

Light component 702 increases near time point 710 due to the increased ambient light detected during the “on” periods in region 708. Ambient signal 704 also increases near time point 710 due to the increased ambient light detected during the “off” periods in region 708.

Plot 720 includes a logic flag that may be set when a potential probe-off condition is detected. The abscissa axis of plot 720 may be in units of time, and the ordinate axis may be in binary logic values 0 and 1. In some embodiments, flag signal 722 may be indicative of a flag or logic value set when a potential probe-off condition is detected. For example, when an ambient signal characteristic is compared to a threshold in step 508 of FIG. 5, the result of the comparison may be used to set flag signal 722 to 0 or 1. In some embodiments, an ambient signal level above a threshold, may result in flag signal being set to 1.

In the embodiment illustrated in FIG. 7, ambient signal 704 crosses threshold 712. In some embodiments, the system may use a comparison of the levels, trends, other characteristics, or any combination thereof, of ambient signal 704 and light component 702 to determine that ambient signal 704 crossing threshold 712 is not indicative of a probe-off condition (i.e., a false-positive). For example, the system may determine that ambient signal 704 crossing threshold 712 is a false-positive due to a similar change in light component 702. In another example, the system may determine the false-positive due to a rate of change in one or both signals. Plot 720 may indicate that, flag signal 722 is set to 0 in both region 724 before time point 710 and in region 726 after time point 710. It will be understood that any suitable comparison or combination of comparisons of any suitable signals may be used to identify false-positives. For example, an ambient signal rising in combination with a drive pulse signal may indicate a false-positive. In a further example, the duration, magnitude, or occurrence of a threshold crossing may indicate a false-positive. In a further example, a number of threshold, crossings may be indicative of a false-positive. In some embodiments, the system may enter a reset period and/or adjust a threshold following a false-positive. In some embodiments, the system may generate an indication (e.g., visual or audial) that a false-positive has occurred. In some embodiments, a system tolerance for false positives may be user selectable or otherwise adjustable depending on, for example, the condition of the patient. For example, a system may be configured so that any threshold crossing triggers a flag signal. In a further example, a system may be configured so that, a threshold must be crossed for a certain amount, of time or by a certain amount to trigger a flag signal.

It will be understood that, the time intervals indicated by regions 706, 708, 724, and 726 are both approximate and exemplary. Similarly, the particular location of time point 710 is both approximate and exemplary. For example, the boundary between region 706 and region 708 at time point 710 may be slightly earlier or later in time. It will also be understood that the regions may overlap, be non-contiguous, be in any other suitable arrangement, or any combination thereof.

The foregoing is merely illustrative of the principles of this disclosure and various modifications may be made by those skilled, in the art without departing from the scope of this disclosure. The above described embodiments are presented for purposes of illustration and not of limitation. The present disclosure also can take many forms other than those explicitly-described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method for determining whether a physiological sensor is properly positioned on a subject, the method comprising:
using the physiological sensor to emit a photonic signal comprising at least, one wavelength of light;
receiving a light signal, wherein the light signal comprises an ambient light signal component and a component corresponding to the at least one wavelength of light;
determining, using processing equipment, a baseline characteristic of the ambient light signal;
comparing, using the processing equipment, the baseline characteristic of the ambient light signal to a threshold; and
determining, using the processing equipment, that the physiological sensor is not properly positioned based on the comparison.

2. The method of claim 1, wherein the physiological sensor comprises a pulse oximeter sensor.

3. The method of claim 1, wherein using the physiological sensor to emit a photonic signal comprises using at least one light emitting diode.

4. The method of claim 1, wherein receiving a light signal comprises receiving a light signal using a photoelectric detector.

5. The method of claim 1, wherein determining a baseline characteristic of the ambient light signal comprises determining the baseline amplitude of the ambient light signal.

6. The method of claim 1, wherein determining a baseline characteristic of the ambient light signal comprises determining a baseline trend of the ambient light signal.

7. The method of claim 1, wherein the threshold is determined based on the ambient light signal.

8. The method of claim 1, further comprising determining a characteristic of the light signal, wherein determining that the physiological sensor is not properly positioned is further based on the characteristic of the light signal.

9. The method of claim 1, wherein determining that the physiological sensor is not properly positioned comprises determining a probe-off condition.

10. The method of claim 1, further comprising providing an indicator of the determined physiological sensor position.

11. A system for determining whether a physiological sensor is properly positioned on a subject, the system comprising:
an emitter configured to emit a photonic signal comprising at least one wavelength of light;
a detector configured to receive a light signal, wherein the light signal comprises an ambient light signal component and a component corresponding to the at least one wavelength of light; and
processing equipment configured to:
determine a baseline characteristic of the ambient light signal;
compare the baseline characteristic to a threshold; and
determine that the physiological sensor is not properly positioned based on the comparison.

12. The system of claim 11, wherein the physiological sensor comprises a pulse oximeter sensor.

13. The system of claim 11, wherein the emitter comprises a light emitting diode.

14. The system of claim 11, wherein the detector comprises a photoelectric detector.

15. The system of claim 11, wherein the baseline characteristic of the ambient light signal comprises the baseline amplitude of the ambient light signal.

16. The system of claim 11, wherein the baseline characteristic of the ambient light signal comprises a baseline trend of the ambient light signal.

17. The system of claim 11, wherein the threshold, is determined based on the ambient light signal.

18. The system of claim 11, wherein the processing equipment is further configured to:
determine a characteristic of the light signal; and
determine that the physiological sensor is not properly positioned further based on the characteristic of the light signal.

19. The system of claim 11, wherein the processing equipment is further configured, to determine a probe-off condition when the physiological sensor is not properly positioned.

20. The system of claim 11, wherein the processing equipment is further configured to provide an indicator of the determined physiological sensor position.
